(19) United States
(12) Patent Application Publication
(10) Pub. No.: US 2008/0097084 A1
(43) Pub. Date: Apr. 24, 2008

(54) Rho Antibody And Tag To Purify Cell Surface Proteins

(76) Inventors: Michael J. Mendez, Emeryville, CA (US); Craig Behnke, San Diego, CA (US); Craig Hokanson, Pleasanton, CA (US)

Correspondence Address: Novartis Vaccines and Diagnostics, Inc.;Corporate Intellectual Property, P.O. BOX 8097, EMERYVILLE, CA, 94662-8097, US

(21) Appl. No.: 10/573,610

(22) PCT Filed: Sep. 27, 2004
(86) PCT No.: PCT/US04/31831

§ 371 Date: Nov. 26, 2007

Related U.S. Application Data
(60) Provisional application No. 60/506,304, filed on Sep. 25, 2003.

(51) Int. Cl. C07K 1/107; C07K 1/14; C07K 14/00

(52) U.S. Cl. 530/402; 530/350; 530/413

(57) ABSTRACT

A process for the expression and purification of membrane associated proteins is provided. A process for the expression and purification of membrane proteins such as G-protein coupled receptors is provided. A hybrid polypeptide is constructed from a protein of interest and a peptide tag from the amino-terminus extracellular domain of rhodopsin. A method for purifying the hybrid protein using the rhodopsin tag is provided. Methods of detecting the expressed hybrid are also provided.

### FIELD OF THE INVENTION

The invention relates generally to the fields of protein structural biology and pharmaceutical design, and more specifically, to the processes of expression, purification and identification of transmembrane proteins such as G-protein coupled receptors.

### BACKGROUND INFORMATION

Membrane proteins are essential for cellular communication, metabolic regulation, electrical and ion balance, structural integrity of cells, cell adhesion, and other functions. G-protein coupled receptors (GPCRs) are a particularly important family of membrane proteins because they form one of the largest and most functionally diverse groups of receptor proteins. Estimates of the human genome suggest that as much as 2 to 4 percent of the genome encodes GPCRs involved in the regulation of a multitude of physiological process. As a result, drugs directed at GPCRs and other transmembrane proteins are being actively investigated throughout the pharmaceutical industry.

Structural models of membrane proteins have proven useful in predicting mechanisms of ligand binding, predicting the effect of disease-causing mutations, and supporting drug design. However, obtaining structures of membrane proteins at atomic-level resolution is technically challenging, and requires an abundance of homogenous and relatively pure protein.

Unfortunately, natural sources of most membrane proteins are not readily abundant or pure enough to facilitate study. Tissue culture systems have conventionally been used to obtain large amounts of expressed protein; however such systems typically yield only low-levels of recombinant membrane proteins. In particular, recombinant GPCRs are expressed at very low levels in tissue culture. Furthermore, traditional methods of purifying recombinant GPCRs expressed by tissue culture are overly disruptive of the protein structure, resulting in a heterogeneous mixture of protein that is difficult to utilize.

Recombinant membrane proteins are made by first ligating DNA fragments coding for a target protein to an appropriate DNA promoter sequence and ribosome binding site. The resulting recombinant protein may also include a characteristic polypeptide tag useful for identifying and purifying the recombinant protein. The nucleotides encoding the hybrid polypeptide is then inserted into a plasmid expression vector, and the plasmid is transfected into a host prokaryotic or eukaryotic cell. Transformed host cells are identified, isolated and then cultivated based on expression of the desired hybrid protein. Recombinant protein can be purified from the cell culture, possibly by using the included polypeptide tag. For example, a poly-argenine sequence can be added to the protein to allow purification on a cation exchange resin such as SP-Sephadex resin. See U.S. Pat. No. 4,532,207. Another purification technique employs a polyhistidine tag at either the amino- or carboxy-terminus of the hybrid polypeptide. This recombinant protein can then be purified by chromatography on a Ni.sup.2+ metal affinity resin. p Various affinity purification protocols are also currently used to isolate fusion proteins. Affinity chromatography is based on the capacity of proteins to bind specifically and noncovalently with a ligand. This technique can isolate proteins from very complex mixtures with greater purity than sequential ion-exchange and gel column chromatography, and also without significant loss of activity. Typically, a ligand (or tag) that can be bound with high specificity to an affinity matrix is chosen as the fusion partner. For example, the maltose-binding protein domain from the malE gene of E. coli has been used as a fusion partner and allows the affinity purification of the fusion protein on amylose resins.

Epitope tagging is another method used to isolate and detect expression of hybrid polypeptides. Epitope tagging utilizes antibodies against a polypeptide sequence (the “tag”) to label or bind hybrid proteins to which the polypeptide tag has been added. See Kolodziej, P. A. and Young, R. A., Methods Enzymol., 194:508-519 (1991). A short sequence of nucleotides encoding the epitope is inserted into the coding region of the cloned gene, and the hybrid gene is introduced into a cell by a method such as transformation. The result is a chimeric protein containing the epitope as a tag. If the epitope is exposed on the surface of the protein, it is available for recognition by the epitope-specific antibody, allowing the investigator to observe the protein within the cell using immunofluorescence or other immunolocalization techniques. Further, fusion proteins labeled with such epitope tags can be used for purifying proteins by affinity purification techniques. The small size of the epitope tag, which is usually 5-20 amino acids in length, is critical because small tags generally have no effect on the biological function of the tagged protein.

Many types of epitope tags have been used, with c-myc and FLAG® tags being two of the most popular. See Evan et al., Mol Cell Biol. 5:3610-3616 (1985). Generally, these epitopes are fused to the amino or carboxy-terminus of the expressed protein making them more accessible to the antibody for detection and less likely to cause severe structural or functional perturbations. Epitope tagging also offers tremendous time savings over the traditional method of producing an antibody to the specific protein being studied. Epitope tagging products and kits, which include various combinations of peptides, polynucleotides, and antibodies, are currently sold by a number of companies, including Boehringer-Mannheim, Indianapolis Ind.; Berkeley Antibody Company, Berkeley, Calif.; MBL International Corporation, Watertown, Mass.; Novagen, Madison Wis.; IBI, West Haven, Conn. and Life Technologies, Gaithersburg, Md.

However, there are problems when using conventional tagging techniques with transmembrane proteins. First, traditional tags, including epitope tags, result in a low-level of properly folded and expressed integral membrane proteins. The GPCRs are a good example of this problem. In cell culture experiments, most recombinantly expressed GPCRs remains trapped in the endoplasmic reticulum. The characteristic low-levels of expression are likely because of improper insertion into the membrane, leading to improper protein folding or other incorrect post-translational modifications (D. Kratwurst et al, Cell 95 (7): 917-26 (1998)). Thus, recombinant GPCRs isolated from tissue culture by traditional methods are heterogeneous and therefore unsuitable for structural studies.

Further, the purification of membrane proteins such as the GPCRs are especially complex because it is critical that the quaternary structure of the protein not be disrupted by the purification process. Traditional purification techniques using polypeptide tags, for example nickel-column purification or cation exchange columns, are too harsh and will disrupt the natural folding of most transmembrane proteins. Purification by traditional epitope tagging is also difficult and costly because of the very low-levels of expression achieved when using recombinant GPCRs in cell culture.

Thus, there is a need for a method for producing and purifying proteins, and particularly membrane proteins such as GPCRs, in high abundance, purity and homogeneity. Such proteins can be used for structural studies as well as for other research and therapeutic applications. The present invention satisfies this need and provides related advantages as well.

### SUMMARY OF THE INVENTION

This invention is directed to a hybrid polypeptide composed of the amino-terminal extracellular domain of rhodopsin acting as an identification polypeptide and fused to the amino-terminal end of a polypeptide of interest. In some embodiments of this invention the amino-terminal extracellular domain of rhodopsin refers to at least the first 20, but up to the first 35, amino acids of bovine rhodopsin.

In some embodiments of this invention, the polypeptide of interest is a membrane polypeptide. In many embodiments, the polypeptide of interest is a G-protein coupled receptor.

Other embodiments of this invention are directed to a method of purifying a target polypeptide by expressing a hybrid polypeptide in which the amino-terminus of a target protein has been fused to the amino-terminal extracellular domain of rhodopsin. The hybrid polypeptide is then complexed with an antibody specific to the amino-terminal extracellular domain of rhodopsin, and the resulting complex is isolated. Finally the hybrid polypeptide is dissociated from the antibody in the complex.

In some embodiments of this method the antibody specific to the amino-terminal extracellular domain of rhodopsin is directed against the first 15 amino acids of rhodopsin; in some embodiments rhodopsin refers specifically to bovine rhodopsin. This invention also contemplates using a G-protein coupled receptor as the target polypeptide.

The invention also contemplates isolating the antibody/hybrid polypeptide complex using a resin. The resin binds and immobilizes the antibody. This invention also encompasses alternate ways to dissociate the hybrid polypeptide from the antibody to rhodopsin. The hybrid polypeptide can be eluted by pH, salt gradient, or by competition binding with an excess of the epitope peptide of the antibody.

Another embodiment of this invention describes a method of labeling a target polypeptide by creating a hybrid polypeptide in which the amino-terminus of a target polypeptide is fused to the amino-terminal extracellular domain of rhodopsin. This hybrid polypeptide is then detected using a reporting agent by forming a complex with the hybrid polypeptide and an antibody against all or a portion of the amino-terminal extracellular domain of rhodopsin. The reporting agent is either directly conjugated to the antibody or linked by the use of a secondary antibody or binding agent. A reporting agent can be fluorescent, enzymatic or radioactive. This invention contemplates using the first 20 amino acids of bovine rhodopsin as the amino-terminal extracellular domain of rhodopsin, as well as an antibody directed against the first 15 amino acids of bovine rhodopsin as the antibody to rhodopsin.

In some embodiments of this method, the polypeptide of interest is a membrane polypeptide. In many embodiments, the polypeptide of interest is a G-protein coupled receptor.

### DETAILED DESCRIPTION OF THE INVENTION

The present invention overcomes the problems of purification of membrane proteins such as the family of G-protein coupled receptors (GPCRs) by providing an epitope tag derived from rhodopsin. Rhodopsin is a prototypical member of the GPCR family of transmembrane proteins. Thus, this invention takes advantage of the properties of the amino-terminal extracellular region of rhodopsin as both an epitope site (an identification peptide, or tag) and as a peptide that assists in inserting the protein into the cell membrane. The invention is directed towards enhanced expression, purification and detection of target polypeptides, in particular members of the GPCR family of proteins. Fusing the amino-terminus of a target protein to the amino terminal extracellular region of the GPCR protein rhodopsin (RHO) produces a hybrid polypeptide which can be expressed at high levels and which is bound by antibodies to rhodopsin.

The following examples are intended to convey certain principles of the invention. These examples are not intended to limit the scope of the claims to any particular example. It is understood that the claims are to be given their broadest reasonable interpretation in view of the description herein, any prior art, and the knowledge of those of ordinary skill in the field. Those skilled in the art will readily appreciate that many variations may be derived using the following description.

This invention responds to the need to express and purify proteins and polypeptides that span or are associated with the plasma membrane. Target polypeptides are proteins of interest to the user, and include primarily transmembrane proteins such as the GPCR family of proteins. However, this invention is not limited to GPCR proteins, or even to complete (full-length) proteins. For the purpose of this invention, target polypeptide refers to a protein or portion of a protein that spans or associates with the plasma membrane. Since the identification polypeptide (the epitope tag) is derived from rhodopsin, homology among the GPCR family of proteins suggests that this invention is ideal for expressing and purifying members of the GPCR family.

There are many forms of rhodopsin, both within and across species. One of the best-studied rhodopsin molecules is bovine rhodopsin. The first 36 amino acids of the bovine rhodopsin (RHO) protein are extracellular, and may assist in inserting the rhodopsin protein in the membrane. The first 20 amino acids from the amino-terminus of the bovine RHO protein (MNGTEGPNFY VPFSNKTGW) (SEQ ID NO: 1) can be readily incorporated at the amino-terminus of a target polypeptide of interest, and expressed in cell culture. This polypeptide sequence makes a good epitope tag because it is extracellular, and therefore accessible, and also because it is believed to include a cryptic site assisting in membrane insertion. Furthermore, because rhodopsin is a prototypical member of the GPCR family, the RHO tag is unlikely to interfere with processing or folding of other transmembrane proteins. Thus, the amino-terminal portion of bovine rhodopsin also aids in expressing functional tagged protein on the cell membrane.

There are also many antibodies that can specifically recognize the amino-terminal domain of bovine rhodopsin. See D. Hicks and R. S. Molday, Exp. Eye Res. 1986, 42 (1):55-71, P. Rohlich et al, Exp. Eye Res. 1989, 49 (6):999-1013, P. A. Hargrave et al, Exp. Eye Res. 1986, G. Adanus et al, Vision Res. 1991, 31 (1):17-31 and G. Adamus et al, Pept. Res. 1988, 1 (1):4247. Antibodies, particularly monoclonal antibodies, directed against the first 15 amino-terminal peptides of bovine rhodopsin (MNGTEGPNFY VPFSN) (SEQ ID NO: 2) are highly effective for identifying hybrid polypeptides tagged with the first 20 amino acids from the amino-terminus of bovine rhodopsin. However, antibodies directed against the amino-terminal regions of other forms of rhodopsin would also apply to this invention. It is important that the antibody bind with high affinity to the rhodopsin identification peptide portion of the hybrid polypeptide. Usually this will mean that the antibody was directed against all or a portion of the identification peptide.

Alternative embodiments of the invention contemplate using the amino terminal extracellular domains of rhodopsin from any other species. Examples include rat rhodopsin, human rhodopsin, mouse rhodopsin or fruit fly rhodopsin. For the purposes of this invention, the amino terminal extracellular domain of rhodopsin refers to the most amino terminal portion of rhodopsin which is substantially extracellular when expressed endogenously. The amino terminal extracellular domain is not limited to the entire extracellular region of the amino terminal region, but encompasses a portion or subpart of the extracellular region of the amino terminal region.

Purification of the transmembrane proteins is conceptually straightforward. For example, if the target polypeptide is a member of the GPCR family, it is first fused to the rhodopsin identification peptide to create the hybrid polypeptide described above. The hybrid polypeptide is made from the peptide sequence of the GPCR target polypeptide by ligating the nucleotide sequence encoding the GPCR protein with the nucleotide sequence coding for the first 20 amino acids of bovine rhodopsin. The sequences should be joined such that the nucleotides coding the amino-terminus of rhodopsin will be linked to the amino-terminus of the GPCR target polypeptide. The resulting hybrid polynucleotide (coding for the hybrid polypeptide) is then inserted into an expression vector capable of expressing in cultured cells, such as Sigma's FLAG expression vector (Sigma-Aldrich Corp., St. Louis, Mo.). Cultured cells can then be transformed with the plasmid, and induced to express the hybrid polynucleotide.

The expressed protein can be purified using an antibody having a high specificity and affinity for the rhodopsin tag. This example uses a monoclonal antibody raised in mouse and directed against the first 15 amino acids of bovine rhodopsin (SEQ ID NO: 2). The antibody is conjugated to a matrix, such as a resin like sepharose, and a solution containing the hybrid polypeptide harvested from the cultured cells is applied to the matrix. The exposed amino-terminal portion of the hybrid polypeptide binds to the antibody, and is held in the matrix. The resin can then be washed to remove impurities. Finally, the hybrid polypeptide is eluted from the resin. Depending on the resin and desired stringency of elution, different ways of eluting the hybrid polypeptide may be used. For example, the hybrid polypeptide can be eluted by pH gradient, salt gradient or detergent. Alternatively, the hybrid polypeptide could be competed off of the antibody using a small peptide. For this example, an excess of peptide expressing the first 15 amino acids of rhodopsin could be used to elute the hybrid polypeptide described above.

Purified hybrid polypeptide can then be analyzed. This invention is particularly directed at aiding in the creation of protein crystals for protein structure determination by crystallographic techniques. Because the amino-terminal rhodopsin tag is small, it should not alter or complicate the crystal structure determination. Bovine rhodopsin also has the advantage that it has been crystallized, and therefore the structure of the amino-terminal domain is known.

It is also possible to detect the hybrid polypeptide after it has been expressed. For example, the rhodopsin marker can be used to monitor cell-surface expression of hybrid polypeptides such as membrane proteins. A hybrid protein made as described above by fusing a rhodopsin marker (identification peptide) to a GPCR protein can be visualized using an antibody to the RHO tag. Cells transformed by the plasmid containing the hybrid polypeptide can be immunolabled to verify that the proteins are being expressed in the plasma membrane in adequate levels. Since the 20 amino acid tag is expressed extracellularly, it is also potentially useful as a vital marker in living cells, as well as fixed. Further, surface and total expression can be quantified by binding assays with radiolabeled antibodies. Visual immunohistochemistry using the anti-rhodopsin antibody to recognize the rhodopsin tag on the hybrid polypeptide is can also be used to quantify surface or internal expression. Hybridization blotting using an antibody to the rhodopsin tag (e.g. Western blotting) is another way to determine expression of the hybrid polypeptide.

Virtually any reporting agent can be used in conjunction with the antibody against the rhodopsin tag. Enzymatic reporting agents such as horseradish peroxidase (HRP ) would be effective. As described above, radioactive labels, and fluorescent labels are also contemplated. The reporting agent could be either conjugated or incorporated directly into the anti-rhodopsin antibody. Alternatively, the reporting agent could be linked to the antibody/hybrid protein complex through a secondary antibody. If the antibody against the rhodopsin tag was raised in mouse, a secondary antibody against mouse IgG labeled with a reporting agent could be used to visualize the hybrid polypeptide.

We claim:

1. A hybrid polypeptide comprising:
a target polypeptide,
an identification peptide comprising the amino-terminal extracellular domain of rhodopsin, where said identification peptide is linked to the amino-terminus of said target polypeptide.

2. The hybrid polypeptide of claim 1 where said identification peptide comprises an amino terminal extracellular domain of bovine rhodopsin.

3. The hybrid polypeptide of claim 2 where said identification peptide comprises amino-terminal amino acids of bovine rhodopsin selected from the group consisting of: a first 20, a first 25, a first 30, and a first 35 amino acids.

4. The hybrid polypeptide of claim 1 where said target polypeptide comprises a membrane protein.

5. The hybrid polypeptide of claim 4 where said target polypeptide comprises a G-protein coupled receptor protein.

6. A method of purifying a target polypeptide comprising:
expressing a hybrid polypeptide comprising an identification peptide of the amino terminal extracellular domain of rhodopsin fused to the amino terminal region of a target polypeptide,
forming a complex of said hybrid polypeptide with an antibody against said amino terminal domain of rhodopsin,
isolating said complex, and
dissociating said hybrid polypeptide from said antibody.

7. The method of claim 6 where said target polypeptide comprises a G-protein coupled receptor.

8. The method of claim 6 where said identification peptide comprises amino-terminal amino acids of bovine rhodopsin selected from the group consisting of: a first 20, a first 25, a first 30, and a first 35 amino acids.

9. The method of claim 8 where said antibody is directed against a first 15 amino acids of bovine rhodopsin.

10. The method of claim 6 where said antibody is held by a resin.

11. The method of claim 6 where said hybrid polypeptide is dissociated from said antibody by a pH gradient

12. The method of claim 6 where said hybrid polypeptide is dissociated from said antibody by a salt gradient.

13. The method of claim 6 where said hybrid polypeptide is dissociated from said antibody by competing with a peptide comprising of all or a portion of the amino terminal domain of rhodopsin.

14. A method of labeling a target polypeptide comprising:
expressing a hybrid polypeptide comprising: an amino terminal extracellular domain of rhodopsin fused to an amino terminal region of a target polypeptide;
forming a complex of said hybrid polypeptide and an antibody against said amino terminal extracellular domain of rhodopsin; and
detecting said complex by means of a reporting agent.

15. The method of claim 14 where said amino terminal domain of rhodopsin comprises amino-terminal amino acids of bovine rhodopsin selected from the group consisting of: a first 20, a first 25, a first 30, and a first 35 amino acids.

16. The method of claim 15 where said antibody is directed against a first 15 amino acids of bovine rhodopsin.

17. The method of claim 14 where said target polypeptide is a G-protein coupled receptor.

18. The method of claim 14 where said reporting agent is conjugated to said antibody.

19. The method of claim 14 where said reporting agent is a fluorescent marker.

20. The method of claim 14 where said reporting agent is an enzymatic marker.

21. The method of claim 14 where said reporting agent is a radioactive marker.

22. The method of claim 14 where said complex is detected by biding said complex to a secondary antibody against said antibody to the amino terminal extracellular domain of rhodopsin, and said secondary antibody is linked to a reporting agent.
